(19) United States
(12) Patent Application Publication
(10) Pub. No.: US 2022/0071549 A1
(43) Pub. Date: Mar. 10, 2022

(54) APPARATUS, SYSTEMS AND METHODS OF USE FOR OCULAR SURFACE POTENTIAL DIFFERENCE MEASUREMENT

(71) Applicant: The Regents of the University of California, Oakland, CA (US)

(72) Inventors: Alan S. Verkman, San Francisco, CA (US); Julie M. Shallhorn, San Francisco, CA (US); Neel D. Pasricha, San Francisco, CA (US); Marc H. Levin, San Carlos, CA (US)

(21) Appl. No.: 17/469,282

(22) Filed: Sep. 8, 2021

Related U.S. Application Data
(60) Provisional application No. 63/075,759, filed on Sep. 8, 2020.

(51) Int. Cl. A61B 5/398; A61B 3/10; A61B 5/0538

(52) U.S. Cl. CPC A61B 5/398; A61B 5/0538; A61B 3/101

(57) ABSTRACT

The disclosed apparatus, systems and methods relate to ocular surface potential difference (OSPD) measurement, and in particular, to the devices, methods, and design principles allowing for such measurement and the use of the measured OSPD in various research and clinical settings.

### CROSS-REFERENCE TO RELATED APPLICATION(S)

This application claims the benefit under 35 U.S.C. § 119(e) to U.S. Provisional Application 63/075,759, filed Sep. 8, 2020 and entitled “Apparatus, Systems and Methods of Use for Ocular Surface Potential Difference Measurement,” which is hereby incorporated herein by reference in its entirety.

### GOVERNMENT SUPPORT

This invention was made with government support under grant nos. R01 EY013574 and P30 DK072517 awarded by The National Institutes of Health. The government has certain rights in the invention.

### FIELD

The disclosed technology relates generally to ocular surface potential difference (“OSPD”), and in particular, to the devices, methods, and design principles allowing for the use of OSPD in various research and clinical settings.

### BACKGROUND

The cornea and conjunctiva are lined by stratified epithelial cell layers in contact with the tear film. As in other organs, epithelial cells lining the ocular surface express ion transport proteins that can facilitate active fluid secretion or absorption to regulate tear fluid volume and osmolality.

Major ion channels that are functionally expressed in ocular surface epithelial cells include the cystic fibrosis transmembrane conductance regulator (“CFTR”) chloride channel and the epithelial sodium channel (“ENaC”), which are thought to facilitate fluid secretion and absorption, respectively. The ocular surface epithelium is subject to injury in various infectious and inflammatory conditions, such as bacterial keratitis and Sjögren's syndrome, and with various types of trauma, including desiccation and abrasion.

Clinical evaluation of ocular surface health typically involves slit lamp examination of the fluorescein-stained cornea and the lissamine green-stained conjunctiva, as well as measurement of tear breakup time, Schirmer test of tear fluid volume, and corneal sensation. Determinations of tear fluid osmolality and cytokine levels, and cellular composition of ocular surface tissues, may also provide useful data in the evaluation of ocular surface disease. In mice, millivolt (mV) potentials are dependent on CFTR and ENaC activity, enabling mathematical modeling of individual ion transporter activities.

### BRIEF SUMMARY

The epithelium lining the ocular surface, which includes corneal and conjunctival epithelia, expresses the prosecretory chloride channel cystic fibrosis transmembrane conductance regulator (CFTR) and the proabsorptive sodium channel epithelial sodium channel (ENaC). Here, methodology was established to measure the millivolt (mV) potential differences at the ocular surface (OSPD) in human subjects produced by ion transport.

In Example 1, an ocular surface potential difference (OSPD) system, comprising: a perfusion system comprising a perfusion catheter, a measuring electrode configured to contact an ocular surface, and a reference electrode.

In Example 2, the ocular surface potential difference system of Example 1, further comprising a high-impedance amplifier.

In Example 3, the ocular surface potential difference system of Example 1, wherein the perfusion system is configured to perfuse the ocular surface with one or more solutions.

In Example 4, the ocular surface potential difference system of Example 1, further comprising an operations unit.

In Example 5, a method of measuring ocular transporter and channel behavior, comprising: contacting an ocular surface of the subject with a perfusion catheter and a measuring electrode, perfusing the ocular surface with one or more solutions and the compound via the perfusion catheter, and measuring OSPD at the ocular surface via the measuring electrode, wherein a change in OSPD indicates transporter or channel activation or inhibition.

In Example 6, the method of Example 5, further comprising perfusing with an epithelial sodium channel (ENaC) inhibitor.

In Example 7, the method of Example 5, further comprising perfusing with a cystic fibrosis transmembrane conductance regulator (CFTR) activator.

In Example 8, the method of Example 5, further comprising perfusing with an ENaC inhibitor and a CFTR activator.

In Example 9, the method of Example 8, wherein the ENaC inhibitor is selected from the group consisting of amiloride, amiloride analogs and P321.

In Example 10, the method of Example 8, wherein the CFTR activator is selected from the group consisting of: cystic fibrosis potentiators, VX-770, GLPG2451, wildtype CFTR activators, K-089 and Compound 12 (CFTRact-K267).

In Example 11, the method of Example 5, wherein the measuring OSPD comprises measuring ocular surface depolarization and hyperpolarization.

In Example 12, a method for identifying a candidate compound that affects a target when administered to an ocular surface of a subject, comprising: contacting an ocular surface of the subject with a perfusion catheter and a measuring electrode, perfusing the ocular surface with a solution to establish a baseline via the perfusion catheter, perfusing the ocular surface with the candidate compound via the perfusion catheter, and measuring potential differences at the ocular surface via the measuring electrode, wherein a change in the potential differences from the baseline indicates that the compound affects the target, and wherein the target is selected from the group consisting of ion transporters, ion channels, ENaC inhibitors and CFTR activators.

In Example 13, the method of Example 12, further comprising perfusing with an ENaC inhibitor.

In Example 14, the method of Example 12, further comprising perfusing with a cystic fibrosis transmembrane conductance regulator (CFTR) activator.

In Example 15, the method of Example 12, further comprising perfusing with an ENaC inhibitor and a CFTR activator.

In Example 16, the method of Example 12, wherein the candidate compound is a pharmaceutical or a topical ocular therapeutic.

In Example 17, the method of Example 12, wherein the measuring electrode is connected to a high-impedance voltmeter.

In Example 18, the method of Example 12, wherein the measuring OSPD comprises measuring ocular surface depolarization and hyperpolarization.

In Example 19, a method of measuring OSPD in a subject, comprising contacting an ocular surface of the subject with a perfusion catheter and a measuring electrode, perfusing the ocular surface with one or more solutions and the compound via the perfusion catheter, and measuring OSPD at the ocular surface via the measuring electrode.

In Example 20, a method of assessing the integrity of ocular surface epithelia in a subject following injury by measuring OSPD response in the subject.

In Example 21, the method of Example 20, wherein the OSPD response is measured for between about one and about thirty minutes.

In Example 22, a method for identifying a candidate compound that affects a target when administered to an ocular surface of a subject, comprising: contacting an ocular surface of the subject with a perfusion catheter and a measuring electrode, perfusing the ocular surface with a solution to establish a baseline via the perfusion catheter, perfusing the ocular surface with the candidate compound via the perfusion catheter, and measuring potential differences at the ocular surface via the measuring electrode, wherein a change in the potential differences from the baseline indicates that the compound affects the target, and wherein the candidate compound is an ocular therapeutic or pharmaceutical.

In Example 23, a system for assessing ocular surface ion transport, comprising: a perfusion system comprising a perfusion catheter, a measuring electrode configured to contact an ocular surface, and an operations unit, wherein the operations unit is configured to measure OSPD in a subject during perfusion.

In Example 24, an ocular surface diagnostic system, comprising: a perfusion system comprising a perfusion catheter, a measuring electrode configured to contact an ocular surface, and a software module, wherein the software module is configured to measure OSPD in a subject during perfusion, and wherein a change in measured OSPD is indicative of a condition.

In Example 25, a clinical ocular diagnostic system, comprising: a perfusion catheter, and a measuring electrode constructed and arranged to contact an ocular surface and measure OSPD changes at the ocular surface, wherein measured OSPD changes are indicative of an ocular surface condition.

In Example 26, the system of Example 25, wherein the ocular surface condition is a corneal disease.

In Example 27, the system of Example 25, wherein the ocular surface condition is ectasia.

In Example 28, the system of Example 25, wherein the ocular surface condition is keratoconus.

In Example 29, the system of Example 25, wherein the ocular surface condition is an infection.

In Example 30, the system of Example 25, wherein the ocular surface condition is selected from the group consisting of bacterial, fungal and parasitic infections.

In Example 31, the system of Example 25, wherein the ocular surface condition is selected from the group consisting of ocular surface lesions, pterygia, ocular surface squamous neoplasia and conjunctival lymphoma.

In Example 32, a method of evaluating epithelial disruption, comprising: measuring baseline OSPD at the ocular surface, applying an ocular prosthetic to the ocular surface, measuring condition OSPD at the ocular surface in the presence of the ocular prosthetic, and comparing condition OSPD to baseline OSPD.

In Example 33, a method of identifying genetic conditions in a subject, comprising: contacting an ocular surface of the subject with a perfusion catheter and a measuring electrode, perfusing the ocular surface with one or more solutions and the compound via the perfusion catheter, and measuring OSPD at the ocular surface via the measuring electrode, wherein a change in OSPD indicates a genetic condition in the subject.

In Example 34, an ocular surface potential difference (OSPD) system, comprising a perfusion system comprising a perfusion catheter configured to be positionable adjacent to an ocular surface, a measuring electrode operably coupled to the perfusion catheter, a reference needle operably coupled to a reference electrode, and an electrical measurement device operably coupled to the measuring electrode and the reference electrode.

In Example 35, the system of Example 34, wherein the electrical measurement device comprises a voltmeter.

In Example 36, the system of Example 34, wherein the perfusion system is configured to perfuse the ocular surface with one or more solutions.

In Example 37, the system of Example 34, further comprising a processor coupled to the electrical measurement device.

In Example 38, the system of Example 37, wherein the processor is configured to measure OSPD in a subject during perfusion.

In Example 39, the system of Example 37, further comprising a software module associated with the processor, wherein the software module is configured to measure OSPD in a subject during perfusion.

In Example 40, the system of Example 34, wherein the perfusion system further comprises at least two solution delivery devices fluidically coupled to the perfusion catheter.

In Example 41, the system of Example 34, further comprising a positioning device operably coupled to the perfusion catheter.

In Example 42, the system of Example 34, wherein the measuring electrode is constructed and arranged to measure OSPD changes at the ocular surface, wherein measured OSPD changes are indicative of a condition.

In Example 43, the system of Example 42, wherein the condition is an ocular surface condition selected from the group consisting of corneal disease, ectasia, keratoconus, an infection, ocular surface lesions, pterygia, ocular surface squamous neoplasia, and conjunctival lymphoma.

In Example 44, a method of measuring OSPD in a subject, comprising positioning a perfusion catheter in close proximity with an ocular surface of the subject, wherein the perfusion catheter is operably coupled to a measuring electrode, perfusing the ocular surface with one or more solutions via the perfusion catheter, and measuring OSPD at the ocular surface via the measuring electrode.

In Example 45, the method of Example 44, further comprising perfusing the ocular surface with an epithelial sodium channel (ENaC) inhibitor.

In Example 46, the method of Example 44, further comprising perfusing the ocular surface with a cystic fibrosis transmembrane conductance regulator (CFTR) activator.

In Example 47, the method of Example 44, further comprising perfusing the ocular surface with an ENaC inhibitor and a CFTR activator.

In Example 48, the method of Example 47, wherein the ENaC inhibitor is selected from the group consisting of amiloride, amiloride analogs and P321.

In Example 49, the method of Example 47, wherein the CFTR activator is selected from the group consisting of: cystic fibrosis potentiators, VX-770, GLPG2451, wildtype CFTR activators, K-089 and Compound 12 (CFTRact-K267).

In Example 50, the method of Example 44, wherein the measuring OSPD comprises measuring ocular surface depolarization and hyperpolarization.

In Example 51, the method of Example 44, wherein a change in OSPD indicates transporter or channel activation or inhibition.

In Example 52, the method of Example 44, wherein a change in OSPD indicates a genetic condition in the subject.

In Example 53, a method for identifying a candidate compound that affects a target when administered to an ocular surface of a subject, comprising positioning a perfusion catheter in close proximity with an ocular surface of the subject, wherein the perfusion catheter is operably coupled to a measuring electrode, perfusing the ocular surface with a solution to establish a baseline via the perfusion catheter, perfusing the ocular surface with the candidate compound via the perfusion catheter, and measuring potential differences at the ocular surface via the measuring electrode, wherein a change in the potential differences from the baseline indicates that the compound affects the target, and wherein the target is selected from the group consisting of ion transporters, ion channels, ENaC inhibitors and CFTR activators.

In Example 54, the method of Example 53, further comprising perfusing the ocular surface with an ENaC inhibitor.

In Example 55, the method of Example 53, further comprising perfusing the ocular surface with a cystic fibrosis transmembrane conductance regulator (CFTR) activator.

In Example 56, the method of Example 53, further comprising perfusing the ocular surface with an ENaC inhibitor and a CFTR activator.

In Example 57, the method of Example 53, wherein the candidate compound is a pharmaceutical or a topical ocular therapeutic.

In Example 58, the method of Example 53, wherein the measuring electrode is connected to a voltmeter.

In Example 59, the method of Example 53, wherein the measuring OSPD comprises measuring ocular surface depolarization and hyperpolarization.

While multiple embodiments are disclosed, still other embodiments of the disclosure will become apparent to those skilled in the art from the following detailed description, which shows and describes illustrative embodiments of the disclosed apparatus, systems and methods. As will be realized, the disclosed apparatus, systems and methods are capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

FIG. 1 is a schematic of major ion transporters in human ocular surface epithelia. ENaC, epithelial sodium channel; CFTR, cystic fibrosis transmembrane conductance regulator (chloride channel); CaCC, calcium-activated chloride channel; NKCC1, sodium-potassium-chloride cotransporter, Na/K ATPase, sodium-potassium pump. Transepithelial chloride secretion onto the ocular surface requires an electrochemical driving force to transport chloride from cell cytoplasm onto the ocular surface through chloride channels CFTR and/or CaCC. The electrochemical driving force is established by concerted action of K⁺ channels, NKCC1, and the Na/K ATPase.

FIG. 2A depicts a method for measurement of OSPD in human subjects. FIG. 2A is a schematic showing the multi-syringe perfusion system that delivers fluid to bathe a portion of the ocular surface, and the electrical system with measuring electrode in contact with the ocular surface (through the perfusate), subcutaneous reference electrode, and high-impedance amplifier. The subject's head is stabilized using a slit lamp and the tip of the perfusion catheter is positioned in a fluid pool near the ocular surface using a 3-axis micromanipulator during slit-lamp visualization.

FIG. 2B depicts an OSPD measurement study, showing an operator positioning the tip of perfusion catheter and an assistant operating the perfusion system.

FIG. 2C depicts a photograph of a perfusion catheter in a fluid pool created by eversion of the lateral lower eyelid.

FIG. 3 depicts an OSPD measurement in a non-CF subject. Original recording of OSPD from subject #3, showing OSPD over time in response to serial perfusate solution exchanges as indicated.

FIG. 4A depicts OSPD values deduced from experiments as in FIG. 3. Absolute OSPD values from six non-CF subjects with normal physiological saline solution (‘High Cl⁻’, Solution #1), amiloride-containing normal physiological saline solution (‘Amiloride’, Solution #2), zero chloride solution with amiloride (‘Zero Cl⁻’, Solution #3), and zero chloride solution with amiloride and isoproterenol (Isoproterenor, Solution #4). Data shown as box-and-whisker plot.

FIG. 4B depicts changes in OSPD (A OSPD) in response to indicated solution changes in non-CF subjects.

FIG. 4C depicts A OSPD in response to isoproterenol comparing non-CF subjects and two CF subjects. * p<0.05, ** p<0.01, *** p<0.001 by two-tailed Student's t-test.

### DETAILED DESCRIPTION

The various embodiments here relate to the reliable electrophysiological measurement of the potential difference (PD) across the ocular surface (“OSPD”), the systems and methods for said measurement, and the resulting unique functional data regarding the physiology of the human ocular surface for potential use and various applications in ocular health and disease. For example, according to certain implementations, the measured potentials can provide a functional assessment of the ocular surface and broad applications in ocular health and disease. Further, the resulting data allows for investigation of the in vivo function of CFTR and ENaC at the human ocular surface.

The various embodiments disclosed or contemplated herein relate to devices, systems and methods relating to the use of ocular surface potential difference (OSPD) in clinical, research and diagnostic implementations. For example, in various implementations, the OSPD can be used to evaluate candidate compounds and therapies for use in targeting ocular surface ion transporters. Further, other embodiments relate to assessing the efficacy of topical and systemic drugs acting on ocular surface fluid secretion. In other implementations, the various embodiments relate to the assessment of existing or orphan drugs. Alternative embodiments relate to the study of pharmacodynamics.

In various implementations, the technologies disclosed or contemplated herein relate to the use of CFTR and CaCC activators and the like, ENaC inhibitors, and modulators of signaling with secondary effects on secretory and absorptive mechanisms.

Certain implementations relate to the testing of topical ocular therapeutics, both for direct ocular therapies and for unrelated ocular diseases to assess safety for any indication and to determine effects on integrity of ocular surface epithelia and possible effects on surface transport mechanisms.

Various implementations relate to monitoring the restoration of ocular surface epithelia following injury by assessing OSPD response, which is indicative of normal function. That is, the disclosed implementations demonstrate that OSPD is able to provide functional information to add to existing methods such as fluorescein staining. Examples include desiccation, corneal abrasion, refractive surgery, corneal erosions, neurotropic keratopathy, trauma, ulceration and the like.

Certain implementations of the disclosed technologies are used to study mechanisms of ocular surface fluid transport in research and development. Certain of these implementations utilize OSPD to identify novel therapeutic targets for ocular surface diseases such as dry eye and other conditions. Various implementations also relate to diagnosing genetic disorders involving defective ion or fluid transporters, including but not limited to cystic fibrosis, as would be readily appreciated. Further implementations of the disclosed technologies utilize OSPD to evaluate the safety and/or epithelial disruption of ocular prosthetic devices such as contact lenses and the like. Further examples are of course possible.

Certain implementations of the disclosed technologies are used to assess corneal disease status and recovery in various conditions. In certain of these implementations, OSPD is used to assess ectasia risk, keratoconus progression and the like. Certain implementations of the disclosed technologies are used to assess microbial infections, such as bacterial, fungal and parasitic infections. In certain of these implementations, OSPD is used to assess ocular surface lesions, such as pterygia, ocular surface squamous neoplasia, conjunctival lymphoma and the like. Certain implementations utilize OSPD to assess corneal transplant function.

Other embodiments relate to a clinical device for performing OSPD on clinical subjects for diagnostic purposes. Various alternative implementations feature an operations unit used to assess OSPD response to various perfusions as a tool for assessing the ocular behavior of a clinical subject.

In various implementations, certain ENaC inhibitors are utilized, with certain non-limiting examples being amiloride and amiloride analogs as well as P321 and other known inhibitors. Many other known examples are possible and contemplated. According to other embodiments, certain CFTR activators are utilized, with certain non-limiting examples including cystic fibrosis potentiators such as VX-770 and GLPG2451 and the like, as well as known activators of wildtype CFTR such as K-089, Compound 12 (CFTRact-K267) and the like. Many other known examples are possible and contemplated.

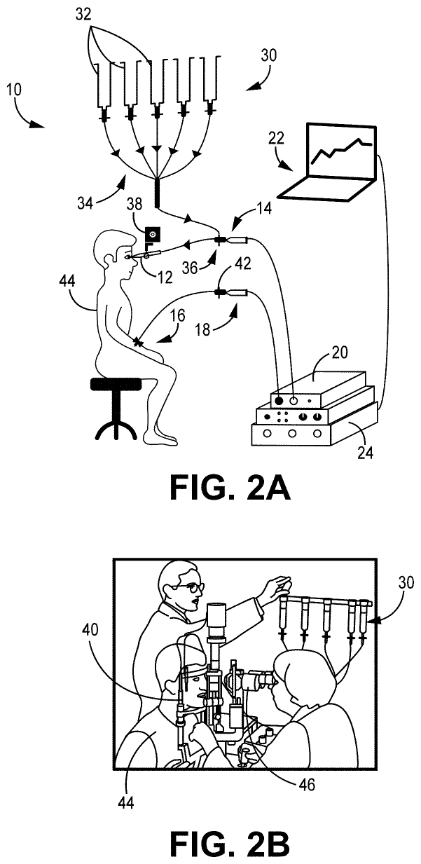

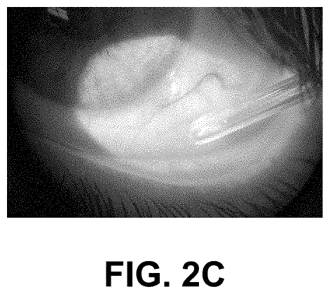

Various systems and devices can be used to measure the potential difference across the ocular surface of a subject. One embodiment of such a system 10 is depicted in FIGS. 2A and 2B. The system 10 has a perfusion catheter 12 coupled to a measuring electrode 14 and a subcutaneous reference needle 16 coupled to a reference electrode 18. The system 10 also has an electrical measurement device 20 coupled to the two electrodes 14, 18 and a computer 22 coupled to the system 10. The measurement device 20 can be a voltmeter and amplifier. Alternatively, any known measurement device can be used. In various embodiments, the computer 22 can be any controller 22 with a processor or any known processor 22 for use in controlling a measurement system. In certain alternative implementations, the system 10 also has a converter 24 that is an analog-to-digital converter 24, with the computer 22 coupled directly to the converter 24 as shown. Alternatively, no converter is required, and the computer 22 is coupled directly to the measurement device 20.

The system 10 also has a perfusion system 30 coupled to the perfusion catheter 12. In the exemplary embodiment as shown, the system 30 has five solution delivery devices 32 coupled to a multiport tubing system 34 that is coupled to the perfusion catheter 12. In the depicted implementation, the delivery devices 32 are syringes 32. Alternatively, any known devices for delivery of solutions for perfusion can be incorporated into the perfusion system 30. Further, while the current perfusion system 30 embodiment has five delivery devices 32, any number of delivery devices 32 can be used (including one, two, three, four, six, seven, eight, nine, ten, or any other number), based upon the desired number of different solutions to be delivered to the perfusion catheter 12. In addition, according to various alternatives, any known perfusion system for delivering various different solutions to the perfusion catheter 12 can be incorporated into the system 10. In one implementation, the delivery devices 32 deliver the perfusion solutions at a rate of 5-10 mL/min. Alternatively, they can deliver the solutions at any known rate.

As shown in FIG. 2A, in accordance with certain embodiments, the multiport tubing system 34 is coupled to the perfusion catheter 12 via a three-way stopcock or port 36. The port 36 couples the perfusion system 30, the perfusion catheter 12, and the measuring electrode 14 as shown. Alternatively, any known coupling port or device can be used to couple the three components.

Various implementations of the system 10 also include a positioning device 38 for precise positioning of the perfusion catheter 12. More specifically, in certain embodiments, the positioning device 38 precisely positions the perfusion catheter 12 in the desired location adjacent to the target eye of the subject, as will be discussed in additional detail below. The positioning device 38 can be a known micromanipulator, such as the 3-axis micromanipulator commercially available from Thorlabs in Newton, N.J. Alternatively, any known precision positioning device can be incorporated into the system 10.

In accordance with certain embodiments as best shown in FIG. 2B, the system 10 can also include a subject stabilization device 40 for maintaining the head (and thus the target eye) of the subject in a substantially fixated position during the measurement procedure using the system 10. For example, the stabilization device 40 can be a known chin rest 40. Alternatively, any known stabilization device can be used.

In addition, the system 10 according to some implementations can include a known slit lamp 46 (as best shown in FIG. 2B) that can be used by the person performing the measurement to position the perfusion catheter 12. Alternatively, any known instrument can be used to visualize the positioning of the catheter 12.

The measurement device 20 can be any known device for use in such measurement systems. For example, in one exemplary embodiment, the device 20 is a BMA-200 high-impedance amplifier/voltmeter, commercially available from ADInstruments in Colorado Springs, Colo. Alternatively any known measurement device can be incorporated into the system 10. Further, the analog-to-digital converter 24 can be any known converter for use in such systems. For example, in one exemplary embodiment, the converter 24 is a PowerLab analog-to-digital converter, also commercially available from ADInstruments.

The perfusion catheter 12 can be any known perfusion catheter for use in such perfusion procedures. For example, in one exemplary embodiment, the perfusion catheter 12 is a commercially available perfusion catheter from Fischer Scientific in Waltham, Mass.

In one embodiment, the measuring electrode 14 and reference electrode 18 are known calomel electrodes. Alternatively, any known electrodes for use in such measurement systems can be used. In accordance with certain implementations, the electrodes 14, 18 are electrically coupled to the perfusion catheter 12 and subcutaneous reference needle 16 using agar. That is, agar is melted and poured into the electrodes 14, 18 and further into the port 42 of the needle 16. In this embodiment, the agar is used as a porous internal support to prevent significant flow of liquids through the tubing, without interfering with electrical transmission. Alternatively, any porous internal support can be used, or no porous support.

According to certain implementations, the five solution delivery devices 32 are 60 mL syringes 32. Alternatively, each of the delivery devices 32 can hold any amount of solution for delivery to the perfusion catheter 12. In some embodiments, the delivery devices 32 are attached to a height-adjustable column or stand that allows for adjustment of the height of the devices 32 in relation to the subject and/or the perfusion catheter 12 to allow for gravity perfusion.

In use, as shown in FIGS. 2A and 2B, a subject 44 is positioned in close proximity to the system 10 prior to the measurement procedure and their head is stabilized using a stabilization device 40. The reference needle 16 is inserted into the arm of the subject 44. In certain embodiments, the lower eyelid of the target eye is everted and fixed in place with an adhesive strip or other similar means to create a receptacle that will allow for pooling of the perfusion solution(s) at the ocular surface.

Once the subject's head is stabilized and the lower eyelid fixed in its everted position, the tip of the perfusion catheter 12 is positioned in the receptacle area created by the lower eyelid. More specifically, in certain embodiments, an operator uses the positioning device 38 and a visualization device 46 to guide the tip of the catheter 12 into the desired position without contacting the ocular surface.

Once the catheter 12 is positioned as desired, the one or more perfusion solutions can be delivered to the receptacle area. If more than one solution is to be delivered, then the separate solutions are contained in separate syringes 32 and each solution is delivered serially: first one, then another, etc. According to one embodiment, each solution is perfused onto the ocular surface fora period of time ranging from about 1 minute to about 20 minutes or until a stable OSPD reading is obtained. Alternatively, the period of time can range from about 1 minute to about 10 minutes. In a further alternative, the period of time can range from about 1 minute to about 3 minutes. In yet another alternative, the period of time can range from 1 minute to any time period between 3 minutes and 20 minutes. Further, the time period can be longer than 20 minutes if an inhibitor/agonist is used that has very slow onset of action.

With the measuring electrode 12 immersed in (and thus in electrical contact with) the solution contacting the ocular surface and the reference electrode 16 inserted subcutaneously into the subject's forearm, the measurement device 20 coupled to the electrodes 12, 16 receives the electrical signals from the electrodes 12, 16. As such, the measurement device 20 thereby measures the electrical potential generated by the ocular surface epithelium.

### Example

The following examples are put forth so as to provide those of ordinary skill in the art with a complete disclosure and description of how the articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

### Methods

OSPD was measured in human subjects in which a fluid-filled measuring electrode contacted a continuously perfused, ˜200 μL fluid pool created by eversion of the lateral lower eyelid, with reference electrode placed subcutaneously in the forearm. OSPD was measured continuously using a high-impedance voltmeter over 10-15 minutes in response to a series of perfusate fluid exchanges.

Human Subjects.

This demonstrative Example was HIPAA-compliant, approved by the University of California San Francisco (UCSF) Institutional Review Board, and adhered to the tenants of the declaration of Helsinki. Written informed consent was obtained from all study subjects. Six non-CF subjects were health care personnel recruited through the UCSF Department of Ophthalmology and two CF subjects with non-functional CFTR mutations (N1303K/Q1100P and W1282X/W1282X) not on CFTR modulator therapy were recruited from the UCSF Cystic Fibrosis Clinic. Exclusion criteria included pediatric age, presence of ocular surface disease on slit lamp examination, history of ocular surgery, current topical eye drop use, or clinically significant allergic rhinitis, ocular allergies, or upper respiratory infection within 30 days. All subjects were given the Ocular Surface Disease Index (OSDI) questionnaire, a validated 12-item scale graded 0-100 to assess for symptoms related to dry eye disease and their effect on vision.

Perfusion Solutions.

The compositions of perfusion solutions (Table 1, below) follow the solutions used in the standardized human nasal potential difference protocol. 13 Solutions #1-3 were made in 1 liter batches, pH balanced to 7.4, and filtered in a sterile environment prior to refrigeration (stable for 3 months). Solutions #4 and #5 (containing isoproterenol or ATP) were made within 2 hours of OSPD measurement. All chemicals were purchased from Sigma-Aldrich (St. Louis, Mo.).

TABLE 1
Perfusate solution compositions

| Solution Number | Solution Name | Solution Contents |
|---|---|---|
| 1 | high Cl⁻ | Buffered Ringer's solution |
| 2 | amiloride | Buffered Ringer's solution + 100 μM amiloride |
| 3 | zero Cl⁻ | Buffered zero chloride solution + 100 μM amiloride |
| 4 | isoproterenol | Buffered zero chloride solution + 100 μM amiloride + 10 |
| | | μM isoproterenol |
| 5 | ATP | Buffered zero chloride solution + 100 μM amiloride + 10 |
| | | μM isoproterenol + 100 μM ATP |

The buffered Ringer's solution contains 1 L Ringer's injection (containing 147.16 mM NaCl, 2.24 mM CaCl₂.2H₂O, and 4.02 mM KCl), 2.41 mM K₂HPO₄, 0.37 mM KH₂PO₄, and 1.18 mM MgCl₂.6H₂O. The buffered zero chloride solution contains 1 L ddH₂O, 2.41 mM K₂HPO₄, 0.37 mM KH₂PO₄, 147.71 mM Na gluconate, 1.22 mM MgSO₄.7H₂O, 4.06 mM K gluconate, and 2.26 mM Ca gluconate.

OSPD Instrumentation.

The electrical components of the instrumentation include measuring and reference electrodes connected to an ISO-Z headstage, BMA-200 high-impedance amplifier/voltmeter, and PowerLab analog-to-digital converter (ADInstruments; Colorado Springs, Colo.) connected to a computer. A perfusion system delivered specified solutions to a perfusion catheter (Fischer Scientific; Waltham, Mass.) whose tip was positioned in a fluid pool at the ocular surface.

To create the reference and measuring electrodes, 3% agar in Ringer's solution was melted and poured into the calomel electrodes. The melted agar-Ringer's mixture was also injected into the Luer lock end of a 23-gauge butterfly needle to create the subcutaneous agar bridge, which was stored in sterile Ringer's solution at room temperature for up to 24. Just prior to testing, offset zeroing was done in a bath containing solution #1 with the reference electrode connected to the agar bridge and the measuring electrode connected to the perfusion catheter.

For solution perfusion, a set of five 60 mL syringes, each with stopcocks, was connected via a multiport tubing system to deliver solutions to a single perfusion catheter. The syringe set was positioned on a height-adjustable column for gravity perfusion at a rate of 5-10 mL/min. Each syringe contained a different perfusion solution at room temperature, which has been shown to produce reliable results in nasal potential difference studies. 15 A three-way stopcock was used to connect the perfusion, measuring electrode, and multiport tubing. The perfusion system was flushed in reverse starting with solution #5 and ending with solution #1.

OSPD Measurement.

The subject was comfortably positioned in front of a slit lamp with their head stabilized on a chin rest. Absorbent gauze pads were secured with paper tape to the subject's cheek to absorb perfusate overflow. The 23-gauge butterfly needle agar bridge connected to the reference electrode was inserted subcutaneously in the forearm. One drop of 0.5% proparacaine was instilled into the test (left) eye for anesthesia. Steri-Strips (3M; Saint Paul, Minn.) were used to evert the lateral lower eyelid to create a ˜200 μL fluid pool. Using a 3-axis micromanipulator (Thorlabs; Newton, N.J.) fixed to the slit lamp, the perfusion catheter tip was guided under direct slit lamp visualization into the inferior fornix and viewed during the OSPD measurement to ensure adequate contact with the fluid pool without contacting the ocular surface. Each solution was perfused onto the ocular surface for 1-3 minutes until a stable OSPD reading was obtained.

Safety.

At the end of the OSPD measurement, lissamine green and fluorescein were applied topically to generate an ocular staining score (OSS) ranging from 0-12.6 For one (non-CF) subject, best-corrected visual acuity (BCVA) and intraocular pressure (IOP) were measured before and just after the session. All subjects received lubricating ophthalmic ointment at the end of the session and were asked to report any subjective ocular surface discomfort at that time and again 24 hours later.

Data Analysis.

OSPD values after each perfusion solution were calculated as the mean value of a 10-second interval at the end of the solution perfusion, as standardized in human nasal potential difference measurements. Data are expressed as mean±S.E.M. Statistical comparisons were made using two-tailed Student's t-test in Microsoft Excel (Microsoft; Seattle, Wash.).

### Results

Summary.

Baseline OSPD in six normal human subjects was −21.3±3.6 mV (S.E.M.). OSPD depolarized by 1.7±0.6 mV following addition of the ENaC inhibitor amiloride, hyperpolarized by 6.8±1.5 mV with a zero chloride solution, and further hyperpolarized by 15.9±1.6 mV following CFTR activation by isoproterenol, a beta-1 and beta-2 adrenergic receptor agonist. The isoproterenol-induced hyperpolarization was absent in two cystic fibrosis subjects lacking functional CFTR. OSPD measurement produced minimal epithelial injury at the ocular surface as assessed by fluorescein and lissamine green staining.

Determinants of the OSPD.

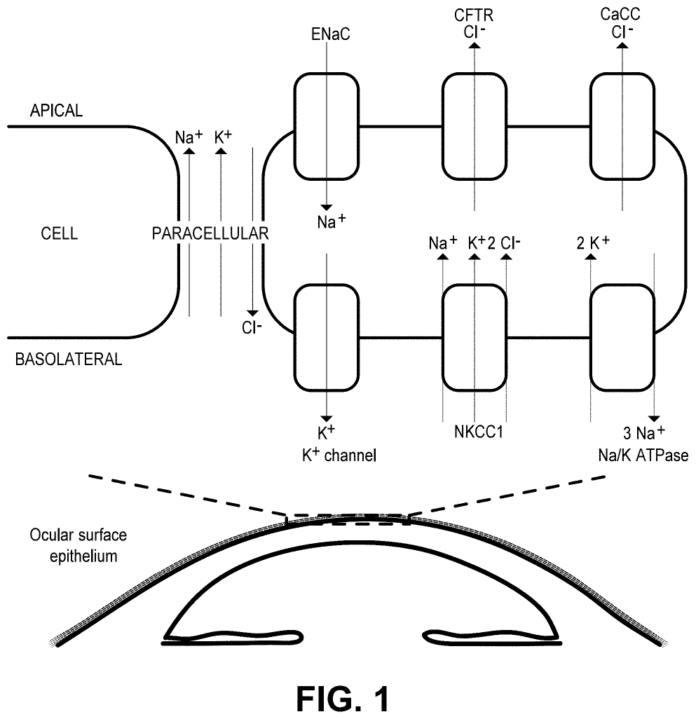

The OSPD is created by the actions of the primary ion transporters expressed in the ocular surface epithelium, a model of which is shown in FIG. 1. These ion transporters are major determinants of tear fluid balance and corneal hydration, among other regulatory aspects. The apical membrane (in contact with tear fluid), expresses the prosecretory, cAMP-activated chloride channel CFTR and calcium-activated chloride channel(s) (CaCC), as would be understood. Further, the basolateral membrane (facing the corneal stroma) contains potassium channels, an electroneutral sodium-potassium-chloride cotransporter (NKCC1), and a sodium-potassium pump (Na/K ATPase), the latter providing the energy to drive fluid secretion.

Paracellular ion transport occurs as well. To create the electrochemical driving force for apical chloride secretion, and corresponding fluid secretion, the basolateral membrane transporters act in concert to maintain a cell interior-negative membrane potential and, in cytoplasm, a high concentration of potassium, a low concentration of sodium, and a concentration of chloride that is above its electrochemical equilibrium potential for its transport onto the ocular surface when CFTR or CaCC are open. The OSPD is negative at the ocular surface as referenced to the corneal stroma.

OSPD Measurement in Humans.

A high-impedance voltmeter measures the electrical potential generated by the ocular surface epithelium, with the measuring electrode immersed in fluid contacting the ocular surface and the reference electrode inserted subcutaneously in the forearm, as is shown for example in FIG. 2A. The measuring electrode makes electrical contact with the ocular surface using a perfusion catheter whose tip is inserted into a small fluid pocket created by eversion of the lateral lower eyelid, as shown in FIG. 2B. Solution exchange is accomplished using a gravity perfusion system. The subject's head is stabilized using a slit lamp, with the tip of the perfusion catheter positioned under direct visualization in the fluid pocket without contacting ocular surface tissue, as shown for example in FIG. 2C.

Robust CFTR Activity at the Human Ocular Surface.

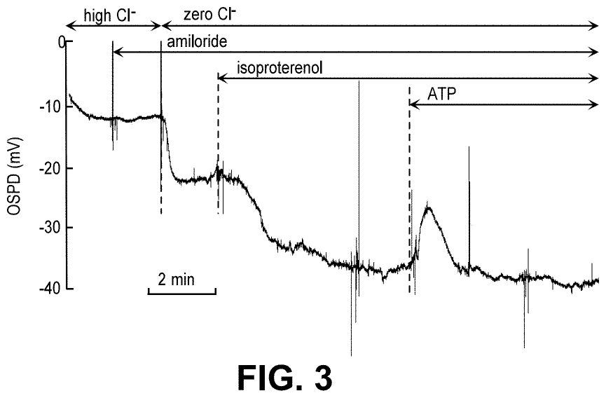

A total of six healthy non-CF subjects were studied, as well as two CF subjects as controls for CFTR function (Table 2, below). FIG. 3 shows a representative recording of OSPD in a non-CF human subject. At the start of the recording there was an initial stabilization period, generally under 1 minute. There was less than 2 mV fluctuation in OSPD with no systematic electrical drift during continuous perfusion with Solution #1, a physiological solution containing high chloride that approximates tear composition. The baseline OSPD in Solution #1 was −21.3±3.6 mV in the six non-CF subjects.

TABLE 2
Clinical characteristics of study subjects

| | Age | | | | | LG | F | Total |
|---|---|---|---|---|---|---|---|---|
| Subject | (Years) | Sex | Race | Ethnicity | OSDI | OSS | OSS | OSS |
| Non-CF | | | | | | | | |
| subjects | | | | | | | | |
| 1 | 38.3 | M | White | Other | 0 | 2 | 0 | 2 |
| 2 | 66.0 | F | White | Other | 2.5 | 0 | 0 | 0 |
| 3 | 31.2 | F | Black | Other | 0 | 0 | 1 | 1 |
| 4 | 28.0 | M | Other | Other | 2.1 | 0 | 2 | 2 |
| 5 | 30.0 | M | Other | Other | 6.3 | 0 | 1 | 1 |
| 6 | 74.5 | M | White | Other | 0 | 5 | 4 | 9* |
| CF | | | | | | | | |
| subjects | | | | | | | | |
| 1 | 55.1 | F | White | Other | 14.6 | 3 | 0 | 3 |
| 2 | 32.2 | F | Other | Hispanic | 0 | 0 | 0 | 0 |

(*Patient asymptomatic, total OSS was 0 the day after initial examination.)

In the table, “OSDI” means ocular surface disease index, “OSS” means ocular staining score, “Total OSS” is the sum of LG+F+extra points, “LG” means lissamine green, and “F” means fluorescein.

After determination of baseline OSPD, four solution exchanges were done to isolate ENaC, CFTR and CaCC functions, as shown in FIG. 3.

Solution #2, a high-chloride solution containing the ENaC inhibitor amiloride, produced minimal depolarization, suggesting minimal ENaC activity.

Solution #3, a zero chloride solution that probes basal transcellular and paracellular chloride transport pathways, produced a rapid, modest hyperpolarization.

Solution #4, containing the cAMP agonist isoproterenol, produced a more gradual, but larger hyperpolarization due to activation of CFTR and potentially other cAMP-dependent ion channels.

Solution #5, containing the calcium agonist ATP, produced a biphasic response due to complex actions of transient elevation in cytoplasmic calcium on CaCC and potassium channels.

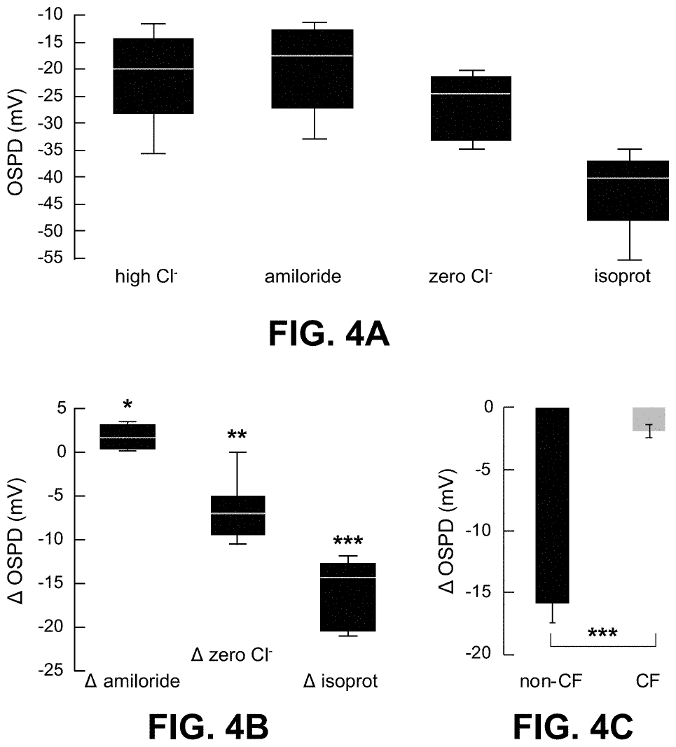

Absolute OSPD values for the six non-CF subjects are summarized in FIG. 4A, with the changes in OSPD (Δ OSPD) produced by the fluid exchanges from solutions #1 to #2, #2 to #3, and #3 to #4 summarized in FIG. 4B. OSPD depolarized by 1.7±0.6 mV following ENaC inhibition by amiloride (solutions #1 to #2), hyperpolarized by 6.8±1.5 mV following exchange from a high to zero chloride solution (solutions #2 to #3), and further hyperpolarized by 15.9±1.6 mV following CFTR activation by isoproterenol (solutions #3 to #4). To confirm that the hyperpolarization induced by isoproterenol was due to CFTR activation, OSPD measurements were done on two CF subjects with CFTR mutations with predicted near-zero CFTR activity. FIG. 4C showed the large isoproterenol-induced hyperpolarization was largely absent in the CF subjects.

Safety.

Several types of studies were done to investigate whether the OSPD procedure caused injury to the cornea or conjunctiva. Total OSS determined immediately following the OSPD procedure was low 3 out of 12) in 7 of the 8 subjects. Most of the staining seen was at the inferotemporal ocular surface where the perfusion was done.

One non-CF subject (subject #6) had a total OSS of 9, though he was asymptomatic and rechecked in clinic the next day with a total OSS of 0. Additionally, this subject had normal BCVA and IOP measure before (20/20-2 and 16 mmHg) and just after (20/20 and 13 mmHg) the OSPD procedure. No subjects reported ocular surface discomfort at the conclusion of the procedure or during the following 24 hours.

Discussion.

We report here the first measurement of the electrical potential generated by the ocular surface epithelium in human subjects, offering a new approach to study ocular surface function and health. This approach was motivated by the experimental use of nasal PD measurements to assess CFTR function in humans with CF, and the development of OSPD in our lab as applied to mice and rabbits. Measurement of OSPD in human subjects is technically straightforward. As discussed further below, the baseline OSPD provides a composite measure of the activities of membrane transport proteins in corneal and conjunctival epithelium. The responses to drugs and ion substitution isolate the activities of specific transport processes.

The technical methods used herein are largely based on prior nasal potential difference measurements in humans and OSPD measurements in small animals, though notable additional developments were needed for OSPD measurement in human subjects. As done for nasal potential difference measurements in humans, an electrical recording system was used that produces accurate OSPD information without significant artifacts, such as junction potentials, and without causing electrical shock, and sterile perfusate solutions were used that contain clinical-grade compounds and approved drugs.

Electrical contact with the ocular surface was accomplished by everting the lower eyelid to create a small fluid pool into which the tip of a soft, flexible perfusion catheter was immersed under direct slit lamp visualization, as opposed to in nasal potential difference studies where the perfusion catheter is blindly inserted into the nostril and cannot be directly visualized in appropriate position with the nasal epithelium, thus causing variable electrical tracings.

The perfusion catheter tip both delivered specified perfusate solutions and maintained electrical contact with the ocular surface. Fluid overflow created by the continuous perfusion was collected using an absorbent gauze secured to the cheek. Various future adaptations and advances are possible, such as development of a custom perfused contact lens system to study cornea versus conjunctiva selectively and eliminate the need for positioning the perfusion catheter tip.

The design of OSPD experiments and the interpretation of data relies on an understanding of the origin of the PD. We previously reported a mathematical model to define quantitatively the influence of the various ion transport processes and paracellular conductance on the OSPD, as well as effects of perfusate ion substitution maneuvers.

The baseline OSPD, which is exterior negative when referenced against the corneal stroma, is the consequence of the active Na/K ATPase at the basolateral membrane of ocular surface epithelial cells. The positive current from the cell interior to the corneal stroma (by exchange of 3 sodium ions for 2 potassium ions) produces, under open-circuit conditions, the exterior negative potential.

The magnitude of the OSPD is affected by the various passive ion transport processes and paracellular resistance. Ion substitution creates a chemical driven force to bias OSPD values to focus on particular sets of ion transport pathways.

For example, the low chloride maneuver used herein, together with ENaC inhibition, enables OSPD values to inform on chloride transport pathways, allowing interpretation of the isoproterenol effect in terms of CFTR activation. While much can be learned by semi-quantitative and comparative OSPD measurements, as has been done for nasal PD measurements, quantitative modeling of the OSPD can enhance data interpretation and identify mechanisms that may not be otherwise apparent.

The OSPD data in the above results demonstrate CFTR as a major prosecretory mechanism in human ocular surface epithelia. A robust average hyperpolarization of 15.6 mV was seen in response to isoproterenol in a zero chloride solution, which was absent in two CF subjects lacking functional CFTR. This cAMP-dependent OSPD hyperpolarization is similar to that seen in human nasal potential difference measurements and in OSPD studies in mice and rabbits. In the animal studies, CFTR-selective inhibitors were also used to confirm that the OSPD hyperpolarization reflects CFTR function, though at present no CFTR inhibitor has been approved for human use. The significant role of CFTR as a prosecretory mechanism at the ocular surface supports the use of CFTR activators as potential therapy for dry eye disorders. A triazine small molecule CFTR activator, which is in preclinical development, has been shown to prevent and reverse dry eye pathology in experimental animal models.

An interesting and perhaps unexpected observation was the minimal effect of amiloride, a blocker of pro-absorptive sodium channel ENaC, on OSPD, with only a 1.7 mV depolarization produced by a high concentration of amiloride. In similar nasal potential difference measurements in humans, amiloride generally produces a >10 mV depolarization, and in mouse and rabbit OSPD measurements amiloride produced 6 and 5 mV depolarizations, respectively. The simplest interpretation of this finding is that ENaC plays a minor role as a pro-absorptive mechanism in human ocular surface, which would suggest that blockers of ENaC, which have been evaluated for dry eye disorders, may have limited efficacy. However, the amiloride data should be interpreted with caution given our incomplete knowledge of the full repertoire of ion transporters in human cornea and conjunctiva.

Measurement of OSPD in human subjects has a number of potential applications in studying basic ocular physiology, evaluating disease status, and testing drug candidates. Changes in OSPD in response to selective modulators of transport and signaling mechanisms, together with ion substitution, are informative in defining transport mechanisms and their regulation, as done here for investigation of ENaC and CFTR. Potassium channels, for example, might be investigated using selective channel modulators and studying effects of potassium ion substitution in the perfusate. OSPD measurements should be informative in quantifying the regulation of ion transport processes in response to disease conditions. For example, whether the expression or function of CFTR is altered in dry eye disorders can be studied, as can potential compensatory upregulation of other prosecretory mechanisms. An intriguing potential application of OSPD is in following the recovery of corneal barrier disruption from a variety of conditions including trauma, infection, and neurotrophic keratopathy. Finally, measurement of OSPD can provide a quantitative surrogate measure of the efficacy and pharmacodynamics of drug candidates that target ion transport mechanisms, such as chloride or potassium channel activators and sodium channel inhibitors.

### CONCLUSION

The disclosed Examples establish the feasibility and safety of OSPD measurement in humans and demonstrate robust CFTR activity, albeit minimal ENaC activity, at the ocular surface. OSPD measurement may be broadly applicable to investigate fluid transport mechanisms and test drug candidates to treat ocular surface disorders.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the term “subject” refers to the target of administration, e.g., an animal. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder.

Although the disclosure has been described with reference to preferred embodiments, persons skilled in the art will recognize that changes may be made in form and detail without departing from the spirit and scope of the disclosed apparatus, systems and methods.

What is claimed is:

1. An ocular surface potential difference (OSPD) system, comprising:
a) a perfusion system comprising a perfusion catheter configured to be positionable adjacent to an ocular surface;
b) a measuring electrode operably coupled to the perfusion catheter;
c) a reference needle operably coupled to a reference electrode; and
(d) an electrical measurement device operably coupled to the measuring electrode and the reference electrode.

2. The ocular surface potential difference system of claim 1, wherein the electrical measurement device comprises a voltmeter.

3. The ocular surface potential difference system of claim 1, wherein the perfusion system is configured to perfuse the ocular surface with one or more solutions.

4. The ocular surface potential difference system of claim 1, further comprising a processor coupled to the electrical measurement device.

5. The ocular surface potential difference system of claim 4, wherein the processor is configured to measure OSPD in a subject during perfusion.

6. The ocular surface potential difference system of claim 4, further comprising a software module associated with the processor, wherein the software module is configured to measure OSPD in a subject during perfusion.

7. The ocular surface potential difference system of claim 1, wherein the perfusion system further comprises at least two solution delivery devices fluidically coupled to the perfusion catheter.

8. The ocular surface potential difference system of claim 1, further comprising a positioning device operably coupled to the perfusion catheter.

9. The ocular surface potential difference system of claim 1, wherein the measuring electrode is constructed and arranged to measure OSPD changes at the ocular surface, wherein measured OSPD changes are indicative of a condition.

10. The ocular surface potential difference system of claim 9, wherein the condition is an ocular surface condition selected from the group consisting of corneal disease, ectasia, keratoconus, an infection, ocular surface lesions, pterygia, ocular surface squamous neoplasia, and conjunctival lymphoma.

11. A method of measuring OSPD in a subject, comprising:
positioning a perfusion catheter in close proximity with an ocular surface of the subject, wherein the perfusion catheter is operably coupled to a measuring electrode;
perfusing the ocular surface with one or more solutions via the perfusion catheter; and
measuring OSPD at the ocular surface via the measuring electrode.

12. The method of claim 11, further comprising perfusing the ocular surface with an epithelial sodium channel (ENaC) inhibitor.

13. The method of claim 11, further comprising perfusing the ocular surface with a cystic fibrosis transmembrane conductance regulator (CFTR) activator.

14. The method of claim 11, further comprising perfusing the ocular surface with an ENaC inhibitor and a CFTR activator.

15. The method of claim 14, wherein the ENaC inhibitor is selected from the group consisting of amiloride, amiloride analogs and P321.

16. The method of claim 14, wherein the CFTR activator is selected from the group consisting of: cystic fibrosis potentiators, VX-770, GLPG2451, wildtype CFTR activators, K-089 and Compound 12 (CFTRact-K267).

17. The method of claim 11, wherein the measuring OSPD comprises measuring ocular surface depolarization and hyperpolarization.

18. The method of claim 11, wherein a change in OSPD indicates transporter or channel activation or inhibition.

19. The method of claim 11, wherein a change in OSPD indicates a genetic condition in the subject

20. A method for identifying a candidate compound that affects a target when administered to an ocular surface of a subject, comprising:
positioning a perfusion catheter in close proximity with an ocular surface of the subject, wherein the perfusion catheter is operably coupled to a measuring electrode;
perfusing the ocular surface with a solution to establish a baseline via the perfusion catheter;
perfusing the ocular surface with the candidate compound via the perfusion catheter; and
measuring potential differences at the ocular surface via the measuring electrode,
wherein a change in the potential differences from the baseline indicates that the compound affects the target, and
wherein the target is selected from the group consisting of ion transporters, ion channels, ENaC inhibitors and CFTR activators.

21. The method of claim 20, further comprising perfusing the ocular surface with an ENaC inhibitor.

22. The method of claim 20, further comprising perfusing the ocular surface with a cystic fibrosis transmembrane conductance regulator (CFTR) activator.

23. The method of claim 20, further comprising perfusing the ocular surface with an ENaC inhibitor and a CFTR activator.

24. The method of claim 20, wherein the candidate compound is a pharmaceutical or an ocular therapeutic.

25. The method of claim 20, wherein the measuring electrode is connected to a voltmeter.

26. The method of claim 20, wherein the measuring OSPD comprises measuring ocular surface depolarization and hyperpolarization.
